(19) United States
(12) Patent Application Publication
(10) Pub. No.: US 2025/0139820 A1
(43) Pub. Date: May 1, 2025

(54) ULTRASOUND IMAGING METHOD, ULTRASOUND IMAGING SYSTEM, AND NON-TRANSITORY COMPUTER-READABLE MEDIUM

(71) Applicant: GE Precision Healthcare LLC, Waukesha, WI (US)

(72) Inventor: Xingti Liu, Wuxi (CN)

(21) Appl. No.: 18/932,217

(22) Filed: Oct. 30, 2024

(30) Foreign Application Priority Data
Oct. 30, 2023 (CN) 202311417828.2

(51) Int. Cl. G06T 7/73; A61B 8/00; A61B 8/08; A61B 90/00; G06F 3/01; G06T 3/40

(52) U.S. Cl. CPC G06T 7/73; A61B 8/4494; A61B 8/461; A61B 8/469; A61B 8/5207; G06F 3/013; G06T 3/40; A61B 2090/372; A61B 2090/378; G06T 2207/10132; G06T 2207/20104; G06T 2207/30004

(57) ABSTRACT

An ultrasound imaging method, including: generating and displaying an ultrasound image of tissue to be imaged; detecting a user's line of sight, and automatically determining the user's region of interest on the ultrasound image; and optimizing ultrasound imaging parameters for the region of interest, and generating and displaying an optimized ultrasound image for the region of interest.

### CROSS-REFERENCE TO RELATED APPLICATION

This application claim priority to Chinese Patent Application No. 202311417828.2, which was file on Oct. 30, 2023 at the Chinese Patent Office. The entire contents of the above-listed application are incorporated by reference herein in their entirety.

### TECHNICAL FIELD

The present invention relates to the field of medical imaging, and in particular, to an ultrasound imaging method, an ultrasound imaging system, and a non-transitory computer-readable medium for automatically optimizing ultrasound imaging parameters.

### BACKGROUND

Ultrasound imaging is a real-time non-invasive imaging technology. It uses an ultrasound probe to transmit ultrasound signals to a site to be imaged and receive echo signals, thereby acquiring ultrasound data about the site to be imaged. By processing the ultrasound data, an ultrasound image of the site to be imaged can be obtained.

In an imaging process, a user needs to adjust ultrasound imaging parameters. There may be several reasons for this. For example, optimal imaging parameters are different for different sites to be imaged. Optimal imaging parameters are different even for different positions at the same site to be imaged. In an actual ultrasound scanning process, a user needs to adjust ultrasound imaging parameters by operating a user interface, to ensure that a desired ultrasound image has satisfactory quality. However, adjustment of ultrasound imaging parameters is difficult. On the one hand, different imaging positions have different preferred imaging parameters, and the adjustment depends on the user's experience, which can be particularly difficult for new doctors. On the other hand, multiple ultrasound imaging parameters affect image quality, making the adjustment highly complex. Moreover, the above process relies heavily on human intervention, making it cumbersome and time-consuming.

### SUMMARY

The aforementioned defects, deficiencies, and problems are solved herein, and these problems and solutions will be understood through reading and understanding the following description.

Some embodiments of the present application provide an ultrasound imaging method, comprising: generating and displaying an ultrasound image of tissue to be imaged; detecting a user's line of sight, and automatically determining the user's region of interest on the ultrasound image; and optimizing ultrasound imaging parameters for the region of interest, and generating and displaying an optimized ultrasound image for the region of interest.

Some other embodiments of the present application provide an ultrasound imaging system, comprising: a probe, configured to acquire ultrasound data about tissue to be imaged; a processor; a display, the display receiving a signal from the processor and performing display; and an image acquisition unit, configured to acquire a user's image and send the image to the processor. The processor is configured to perform the following method: generating and displaying an ultrasound image of tissue to be imaged; detecting a user's line of sight, and automatically determining the user's region of interest on the ultrasound image; and optimizing ultrasound imaging parameters for the region of interest, and generating and displaying an optimized ultrasound image for the region of interest.

In some embodiments of the present application, a non-transitory computer-readable medium is further provided, wherein the non-transitory computer-readable medium has a computer program stored therein, the computer program has at least one code segment, and the at least one code segment is executable by a machine to enable the machine to perform the following method: generating and displaying an ultrasound image of tissue to be imaged; detecting a user's line of sight, and automatically determining the user's region of interest on the ultrasound image; and optimizing ultrasound imaging parameters for the region of interest, and generating and displaying an optimized ultrasound image for the region of interest.

It should be understood that the brief description above is provided to introduce, in a simplified form, concepts that will be further described in the detailed description. The brief description above is not meant to identify key or essential features of the claimed subject matter. The scope is defined uniquely by the claims that follow the detailed description. In addition, the claimed subject matter is not limited to implementations that solve any deficiencies raised above or in any section of the present disclosure.

### BRIEF DESCRIPTION OF THE DRAWINGS

The present application will be better understood by reading the following description of non-limiting embodiments with reference to the accompanying drawings, where:

FIG. 1 is a schematic diagram 100 of an ultrasound imaging system according to some embodiments of the present application;

FIG. 2 is a flowchart of an ultrasound imaging method 200 according to some embodiments of the present application;

FIG. 3 is a schematic diagram 300 of determining a user's region of interest on an ultrasound image according to some embodiments of the present application;

FIG. 4 shows a schematic diagram 400 of optimizing an ultrasound image according to some embodiments of the present application;

FIG. 5 is a schematic diagram 500 of optimizing a volumetric ultrasound image according to some other embodiments of the present application; and

FIG. 6 is a flowchart of an ultrasound imaging method 600 according to some other embodiments of the present application.

### DETAILED DESCRIPTION

Specific embodiments of the present invention will be described below. It should be noted that in the specific description of the embodiments, it is impossible to describe all features of the actual embodiments of the present invention in detail, for the sake of brief description. It should be understood that in the actual implementation process of any embodiment, just as in the process of any one engineering project or design project, a variety of specific decisions are often made to achieve specific goals of the developer and to meet system-related or business-related constraints, which may also vary from one embodiment to another. In addition, it should also be understood that although efforts made in such development processes may be complex and tedious, for a person of ordinary skill in the art related to the content disclosed in the present invention, some design, manufacture, or production changes made on the basis of the technical content disclosed in the present disclosure are only common technical means, and should not be construed as the content of the present disclosure being insufficient.

Unless otherwise defined, the technical or scientific terms used in the claims and the description should be as they are usually understood by those possessing ordinary skill in the technical field to which they belong. “First”, “second”, and similar words used in the present invention and the claims do not denote any order, quantity, or importance, but are merely intended to distinguish between different constituents. The terms “one” or “a/an” and similar terms do not express a limitation of quantity, but rather that at least one is present. The terms “include” or “comprise” and similar words indicate that an element or object preceding the terms “include” or “comprise” encompasses elements or objects and equivalent elements thereof listed after the terms “include” or “comprise”, and do not exclude other elements or objects. The terms “connect” or “link” and similar words are not limited to physical or mechanical connections, and are not limited to direct or indirect connections.

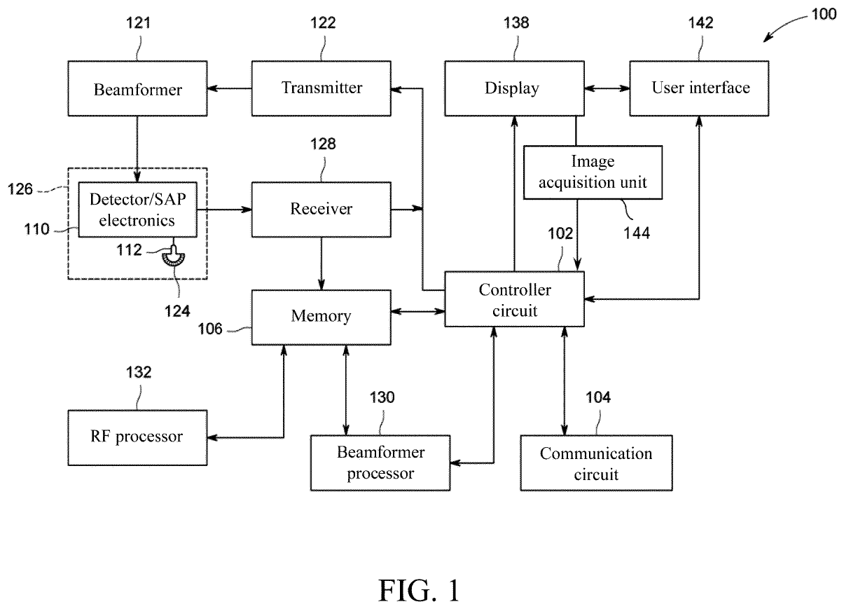

FIG. 1 shows a schematic block diagram of an embodiment of an ultrasound imaging system 100. The ultrasound imaging system 100 may include a controller circuit 102, a display 138, an image acquisition unit 144, a user interface 142, a probe 126, and a memory 106, which can be operatively connected to a communication circuit 104.

The controller circuit 102 is configured to control operation of the ultrasound imaging system 100. The controller circuit 102 may include one or more processors. Optionally, the controller circuit 102 may include a central processing unit (CPU), one or more microprocessors, a graphics processing unit (GPU), or any other electronic component capable of processing inputted data according to a specific logic instruction. Optionally, the controller circuit 102 may include and/or represent one or more hardware circuits or circuit systems, and the hardware circuit or circuit system includes, is connected to, or includes and is connected to one or more processors, controllers, and/or other hardware logic-based apparatuses. Additionally or alternatively, the controller circuit 102 may execute an instruction stored on a tangible and non-transitory computer-readable medium (e.g., the memory 106).

The controller circuit 102 may be operatively connected to and/or control the communication circuit 104. The communication circuit 104 is configured to receive and/or transmit information along a bidirectional communication link with one or more alternate ultrasound imaging systems, remote servers, etc. The remote server may represent patient information, a machine learning algorithm, a remotely stored medical image from a previous scan, and/or a diagnosis and treatment period of a patient, etc. The communication circuit 104 may represent hardware for transmitting and/or receiving data along a bidirectional communication link. The communication circuit 104 may include a transceiver, a receiver, etc., and an associated circuit system (e.g., an antenna) for communicating (e.g., transmitting and/or receiving) with the one or more alternate ultrasound imaging systems, remote servers, etc., by using a wired and/or wireless means. For example, protocol firmware for transmitting and/or receiving data along a bidirectional communication link may be stored in the memory 106 accessed by the controller circuit 102. The protocol firmware provides network protocol syntax to the controller circuit 102 so as to assemble a data packet, establish and/or segment data received along the bidirectional communication link, and so on.

The bidirectional communication link may be a wired (e.g., by means of a physical conductor) and/or wireless communication (e.g., utilizing a radio frequency (RF)) link for exchanging data (e.g., a data packet) between the one or more alternative ultrasound imaging systems, remote servers, etc. The bidirectional communication link may be based on a standard communication protocol, such as Ethernet, TCP/IP, Wi-Fi, 802.11, a customized communication protocol, Bluetooth, etc.

The controller circuit 102 is operatively connected to the display 138 and the user interface 142. The display 138 may include one or more liquid crystal displays (e.g., light emitting diode (LED) backlights), organic light emitting diode (OLED) displays, plasma displays, CRT displays, and the like. The display 138 may display patient information, one or more medical images and/or videos, a graphical user interface, or an assembly received by the display 138 from the controller circuit 102, one or more 2D, 3D, or 4D ultrasound image data sets from ultrasound data stored in the memory 106, or anatomical measurement, diagnosis, and processing information, etc., currently acquired in real time.

The user interface 142 controls the operation of the controller circuit 102 and the ultrasound imaging system 100. The user interface 142 is configured to receive an input from a clinician and/or an operator of the ultrasound imaging system 100. The user interface 142 may include a keyboard, a mouse, a touch pad, a trackball, one or more physical buttons, and the like. Optionally, the display 138 may be a touch screen display that includes at least a portion of the user interface 142. For example, a portion of the user interface 142 may correspond to a graphical user interface (GUI) that is generated by the controller circuit 102 and that is shown on the display 138. The touch screen display may detect the presence of a touch from the operator on the display 138, and may also identify the position of the touch relative to the surface area of the display 138. For example, a user may select, by touching or contacting the display 138, one or more user interface assemblies of the user interface (GUI) shown on the display. User interface assemblies may correspond to icons, text boxes, menu bars, etc., shown on the display 138. A clinician may select, control, and use a user interface assembly, interact with the same, and so on, so as to send an instruction to the controller circuit 102 to perform one or more operations described in the present application. For example, a touch may be applied using at least one among a hand, a glove, a stylus, and the like.

The memory 106 includes a parameter, an algorithm, one or more protocols of ultrasound examination, data values, and the like used by the controller circuit 102 to execute one or more operations described in the present application. The memory 106 may be a tangible and non-transitory computer-readable medium such as a flash memory, a RAM, a ROM, an EEPROM, etc. The memory 106 may include a set of learning algorithms (e.g., a convolutional neural network algorithm, a deep learning algorithm, a decision tree learning algorithm, etc.) configured to define an image analysis algorithm. During execution of the image analysis algorithm, the controller circuit 102 is configured to identify an anatomical feature contained in an ultrasound image. Optionally, an image analysis algorithm may be received by means of the communication circuit 104 along one among bidirectional communication links, and stored in the memory 106. There may be various types of image analysis algorithms, and an exemplary description is provided below.

The image analysis algorithm may be defined by one or more algorithms to identify one or more anatomical features (e.g., a boundary, a thickness, a pixel value change, a valve, a cavity, a chamber, an edge or an inner layer, a vessel structure, etc.) in an ultrasound image. The one or more anatomical features may represent a feature of pixels and/or voxels of the medical image, such as a histogram of oriented gradients, a point feature, a covariance feature, a binary mode feature, and the like. For example, the image analysis algorithm may be defined by using prediction of object identification within the medical image by using one or more deep neural networks.

The image analysis algorithm may correspond to an artificial neural network formed by the controller circuit 102 and/or the remote server. The image analysis algorithm may be divided into two or more layers, such as an input layer for receiving an input image, an output layer for outputting an output image, and/or one or more intermediate layers. Layers of a neural network represent different groups or sets of artificial neurons, and may represent different functions that are executed by the controller circuit 102 with respect to an input image (e.g., an ultrasound image acquired and/or generated by the ultrasound imaging system 100) to identify an object of the input image and determine one or more anatomical features contained in the input image. An artificial neuron in a layer of the neural network may examine an individual pixel in the input image. The artificial neurons use different weights in a function applied to the input image, so as to attempt to identify an object in the input image. The neural network produces an output image by assigning or associating different pixels in the output image with different anatomical features on the basis of the analysis of pixel characteristics.

The image analysis algorithm is defined by a plurality of training images, and the plurality of training images may be grouped into different anatomical planes of interest of the anatomical structure of interest. The training images may represent different orientations and/or cross sections of the anatomical structure of interest corresponding to different fields of view. Additionally or alternatively, the image analysis algorithm may be defined by the controller circuit on the basis of a classification model. The classification model may correspond to a machine learning algorithm based on a classifier (e.g., a random forest classifier, principal component analysis, etc.) configured to identify and/or assign anatomical features to multiple types or categories based on overall shape, spatial position relative to the anatomical structure of interest, intensity, etc.

The controller circuit 102 executing an image analysis algorithm (e.g., an image analysis algorithm) may determine, on the basis of the relationship of the anatomical features relative to each other, modality, etc., anatomical features contained in a current ultrasound image.

Additionally or alternatively, the controller circuit 102 may define a separate image analysis algorithm customized and/or configured for different selected anatomical structures of interest. For example, a plurality of image analysis algorithms may be stored in the memory 106. Each algorithm among the plurality of image analysis algorithms may be customized and/or configured on the basis of different training images (e.g., a set of input images) to configure layers of different neural networks, so as to select anatomical structures of interest, classification models, supervised learning models, and the like.

In some other embodiments, the image analysis algorithm of the controller circuit 102 may also be any other means in the art. For example, the image analysis algorithm may be an algorithm that identifies a boundary of a structure of interest in an image on the basis of changes in an image grayscale value. For example, a certain grayscale value threshold may be set, and neighboring pixel points in the image are compared by using the threshold. If a grayscale value of a neighboring pixel point is greater than the above-described grayscale value threshold, it is determined that the neighboring pixel point is a position to be identified, for example, may be a boundary of a specific anatomical feature. In addition, it should be noted that the example of image identification performed by the controller circuit 102 is described in the foregoing embodiment of the present application, but identification means are not limited thereto, and are not further enumerated herein. For a specific means of use of an identification result of an anatomical feature, an exemplary description is provided below.

With continued reference to FIG. 1, the ultrasound imaging system 100 may include the probe 126, the probe 126 having a transmitter 122, a transmit beamformer 121, and a detector/SAP electronics 110. The detector/SAP electronics 110 may be configured to control switching of transducer elements 124. The detector/SAP electronics 110 may also be used to group the transducer elements 124 into one or more sub-holes.

The probe 126 may be configured to acquire ultrasound data or information from tissues to be imaged (e.g., a fetus, organs, blood vessels, heart, bones, etc.). The probe 126 is communicatively connected to the controller circuit 102 by means of the transmitter 122. The transmitter 122 transmits a signal to the transmit beamformer 121 on the basis of acquisition settings received by the controller circuit 102. The acquisition settings may define the amplitude, pulse width, frequency, gain setting, scanning angle, power, time gain compensation (TGC), resolution, and the like of ultrasonic pulses emitted by the transducer elements 124. The transducer elements 124 emit a pulsed ultrasonic signal into a patient (e.g., the body). The acquisition settings may be defined by a user operating the user interface 142. The signal transmitted by the transmitter 122, in turn, drives a plurality of the transducer elements 124 within a transducer array 112.

The transducer elements 124 transmit a pulsed ultrasonic signal to a body (e.g., a patient) or a volume that corresponds to an acquisition setting along one or more scanning planes. The ultrasonic signal may include, for example, one or more reference pulses, one or more push pulses (e.g., shear waves), and/or one or more pulsed wave Doppler pulses. At least a portion of the pulsed ultrasonic signal is backscattered from tissue to be imaged (e.g., an organ, bone, heart, breast tissue, liver tissue, cardiac tissue, prostate tissue, newborn brain, embryo, abdomen, etc.) to produce an echo. Depending on the depth or movement, the echo is delayed in time and/or frequency, and received by the transducer elements 124 within the transducer array 112. The ultrasonic signal may be used for imaging, for producing and/or tracking a shear wave, for measuring changes in position or velocity within the anatomical structure and a compressive displacement difference (e.g., strain) of tissue, and/or for treatment and other applications. For example, the probe 126 may deliver low energy pulses during imaging and tracking, deliver medium and high energy pulses to produce shear waves, and deliver high energy pulses during treatment.

The transducer elements 124 convert a received echo signal into an electrical signal that can be received by a receiver 128. The receiver 128 may include one or more amplifiers, analog/digital converters (ADCs), and the like. The receiver 128 may be configured to amplify the received echo signal after appropriate gain compensation, and convert these analog signals received from each transducer element 124 into a digitized signal that is temporally uniformly sampled. The digitized signals representing the received echoes are temporarily stored in the memory 106. The digitized signals correspond to backscattered waves received by each transducer element 124 at different times. After being digitized, the signal may still retain the amplitude, frequency, and phase information of the backscattered wave.

Optionally, the controller circuit 102 may retrieve a digitized signal stored in the memory 106 for use in a beamformer processor 130. For example, the controller circuit 102 may convert the digitized signal into a baseband signal or compress the digitized signal.

The beamformer processor 130 may include one or more processors. If desired, the beamformer processor 130 may include a central processing unit (CPU), one or more microprocessors, or any other electronic assembly capable of processing inputted data according to specific logic instructions. Additionally or alternatively, the beamformer processor 130 may execute instructions stored on a tangible and non-transitory computer-readable medium (e.g., the memory 106) to perform beamforming computation using any suitable beamforming method, such as adaptive beamforming, synthetic emission focusing, aberration correction, synthetic aperture, clutter suppression, and/or adaptive noise control, among others. If desired, the beamformer processor 130 may be integrated with and/or be part of the controller circuit 102. For example, operations described as being performed by the beamformer processor 130 may be configured to be performed by the controller circuit 102.

The beamformer processor 130 performs beamforming on the digitized signal of the transducer elements, and outputs a radio frequency (RF) signal. The RF signal is then provided to an RF processor 132 for processing the RF signal. The RF processor 132 may include one or more processors. If desired, the RF processor 132 may include a central processing unit (CPU), one or more microprocessors, or any other electronic assembly capable of processing inputted data according to specific logic instructions. Additionally or alternatively, the RF processor 132 may execute instructions stored on a tangible and non-transitory computer-readable medium (e.g., the memory 106). If desired, the RF processor 132 may be integrated with and/or be part of the controller circuit 102. For example, operations described as being performed by the RF processor 132 may be configured to be performed by the controller circuit 102.

The RF processor 132 may generate, for a plurality of scanning planes or different scanning modes, different ultrasound image data types and/or modes, e.g., B-mode, color Doppler (e.g., color blood flow, velocity/power/variance), tissue Doppler (velocity), and Doppler energy, on the basis of a predetermined setting of a first model. For example, the RF processor 132 may generate tissue Doppler data for multiple scanning planes. The RF processor 132 acquires information (e.g., I/Q, B-mode, color Doppler, tissue Doppler, and Doppler energy information) related to multiple data pieces, and stores data information in the memory 106. The data information may include time stamp and orientation/rotation information.

Optionally, the RF processor 132 may include a composite demodulator (not shown) for demodulating the RF signal to generate an IQ data pair representing an echo signal. RF or IQ signal data may be provided directly to the memory 106 so as to be stored (e.g., stored temporarily). As desired, output of the beamformer processor 130 may be delivered directly to the controller circuit 102.

The controller circuit 102 may be configured to process acquired ultrasound data (e.g., the RF signal data or the IQ data pair), and prepare and/or generate an ultrasound image data frame representing the anatomical structure of interest so as to display the same on the display 138. The acquired ultrasound data may be processed by the controller circuit 102 in real time during a scanning or treatment process of ultrasound examination when echo signals are received. Additionally or alternatively, the ultrasound data may be temporarily stored in the memory 106 during a scanning process, and processed in a less real-time manner in a live or off-line operation. The memory 106 may be used to store processed frames of acquired ultrasound data that are not scheduled to be immediately displayed, or may be used to store post-processed images (e.g., shear wave images and strain images), firmware or software corresponding to, for example, a graphical user interface, one or more default image display settings, programmed instructions, and the like.

Further, the ultrasound imaging system 100 may further include the image acquisition unit 144. The image acquisition unit 144 communicates with the controller circuit 102. The image acquisition unit 144 is capable of acquiring an environment image and sending image data to the controller circuit 102. The processor of the controller circuit 102 is capable of analyzing and processing the environment image. In an example, the processor can identify a user in the image and the direction of the user's line of sight through the obtained environment image. A specific identification manner may be any identification manner in the prior art, and is not limited herein.

The image acquisition unit 144 may be any apparatus having an image acquisition function, e.g., a camera, a 3D camera, a video camera, or the like. In an optional embodiment, the image acquisition unit 144 may be integrated and fixedly arranged on the ultrasound imaging system 100. For example, the image acquisition unit may be arranged on the display 138. With this arrangement manner, in one aspect, it can be ensured that relative positions of the display 138 and the image acquisition unit 144 are fixed, thereby ensuring the accuracy of an algorithm on the ultrasound imaging system 100 for analyzing a direction along which a user is looking at the display 138. In another example, alternatively, the image acquisition unit 144 may be an external device of which a mounting position can be adjusted and calibrated. In addition, there may be multiple image acquisition units 144, and the multiple image acquisition units may operate simultaneously to further ensure that the image data transmitted to the controller circuit 102 is more comprehensive and an analysis result is more accurate. The purpose and advantages of analyzing the image data will be described in detail below.

When performing an ultrasound scan using the ultrasound imaging system 100, the user usually needs to move the probe to a corresponding position of the tissue to be imaged to obtain ultrasound data about the tissue to be imaged. In addition, after reaching the corresponding position, ultrasound imaging parameters further need to be adjusted to acquire an optimal ultrasound image of the tissue to be imaged. There are many types of ultrasound imaging parameters. As enumerated above, the ultrasound imaging parameters may include parameters related to ultrasound data acquisition in an ultrasound imaging process, e.g., the amplitude, the pulse width, and the frequency of an ultrasound pulse, a gain setting, a scanning angle, a power, time gain compensation (TGC), resolution, a frame frequency, etc. The ultrasound imaging parameters may alternatively be parameters related to image processing in the ultrasound imaging process, e.g., parameters related to beamforming such as adaptive beamforming, synthetic transmit focusing, aberration correction, synthetic aperture, clutter suppression, adaptive noise control, and/or the like. In the same tissue to be imaged, different ultrasound imaging parameters may apply to different anatomical features of interest, for example, different imaging parameters apply to anatomical features of different depths or different types. Alternatively, even if some imaging parameters (e.g., parameters such as a frame frequency, resolution, a power, etc.) are suitable for improving image quality for all positions in the same ultrasound image, image quality needs to be compromised in an actual ultrasound imaging process to ensure the safety and stability of the imaging process in consideration of safety issues caused by an upper power limit, heat generation, etc., of the probe. For at least the above reasons, the user, according to experience, needs to make a comprehensive judgment on the current ultrasound imaging, determine preferred ultrasound scanning parameters, and perform manual adjustment in the ultrasound imaging process. On the one hand, this consumes a lot of manpower and time, on the other hand, this presents a great challenge to inexperienced users. Due to the existence of at least the foregoing problems, improvements are proposed in some embodiments of the present application.

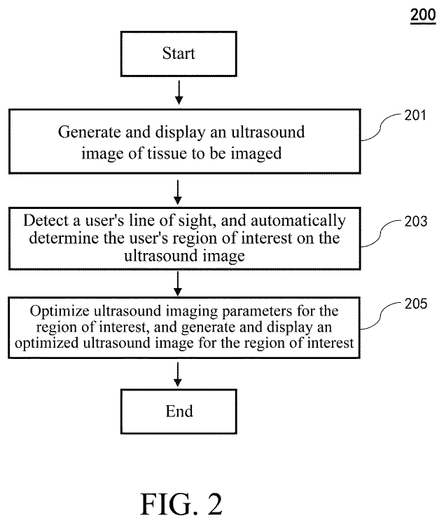

Referring to FIG. 2, this figure shows a schematic diagram of an ultrasound imaging method 200 according to some embodiments of the present application.

In step 201, an ultrasound image of tissue to be imaged is generated and displayed. The process may be implemented by a processor. In particular, the processor may receive ultrasound data of the tissue to be imaged acquired by a probe, and process the data to generate the ultrasound image. Further, the processor further enables the generated ultrasound image to be displayed and to be observable.

In step 203, a user's line of sight is detected, and the user's region of interest on the ultrasound image is automatically determined. In particular, the processor may obtain an image of a current environment, and perform image analysis. The image analysis can determine the user in the environment image and the direction of the user's line of sight. An algorithm and process for the foregoing image analysis may be any algorithm and process in the prior art. For example, facial information and human eye information in the environment image are identified by using any known method, and the orientation of a human eye is comprehensively determined, thereby determining which region of the ultrasound image the user is looking at.

In step 205, ultrasound imaging parameters are optimized for the region of interest, and an optimized ultrasound image for the region of interest is generated and displayed. The ultrasound imaging parameters are parameters related to generation of the ultrasound image. The ultrasound imaging parameters may be one or more of those listed above in the present application, or other parameters related to ultrasound imaging in the art. After determining the region of interest, the processor may automatically perform imaging parameter optimization and configuration for the region of interest, thereby improving the image quality of the region of interest.

With such a configuration manner, it is possible to automatically determine a region for which the user needs to optimize the ultrasound imaging parameters, and to automatically optimize an ultrasound image of the region. In an embodiment of the present application, it is recognized by the inventor that a region for which the user wants to perform image quality optimization is usually highly consistent with a region on which the user's line of sight is focused. On this basis, a region that needs ultrasound imaging parameter optimization can be determined by automatically analyzing and determining the position of the user's line of sight on the ultrasound image. According to the method of the present application, on the one hand, the user does not need to select a specific region to be optimized, which simplifies a workflow and omits an additional operation step of the user. On the other hand, the user also does not need to manually adjust parameters after selecting a specific region to be optimized. Manual parameter adjustment is very cumbersome. Moreover, adjusting many configuration parameters to achieve desired ultrasound image quality for the region to be optimized presents a great challenge to inexperienced practitioners. By using the method of the present application, the workflow is simplified, and it can be ensured that the image quality of the region of interest is quickly improved.

As described above in the present application, there are multiple types of ultrasound imaging parameters. In the field of ultrasound imaging, some ultrasound imaging parameters may affect quality improvement of an entire image. For example, the power of an ultrasound probe, a frame frequency in ultrasound data collection, etc., will affect the definition of an entire ultrasound image, or time gain compensation will affect a display effect of an entire ultrasound image. Some other ultrasound imaging parameters, e.g., adjustment of the position of a focal point, adjustment of a depth, etc., may lead to improvement of ultrasound image quality in some regions and deterioration of ultrasound image quality in other regions.

In an embodiment of the present application, ultrasound imaging parameters being optimized for the region of interest includes: optimizing ultrasound imaging parameters for at least part of the region of interest, and skipping optimizing imaging parameters for at least part of the ultrasound image outside the region of interest. In other words, in the present application, the determination of the region of interest and the optimization of the imaging parameters for the region of interest are both performed in a targeted manner for the region of interest, instead of for the entire ultrasound image. Such a configuration manner includes at least the following advantages: firstly, as some ultrasound imaging parameters cannot improve the image quality of the entire ultrasound image, in the present application, optimizing the ultrasound imaging parameters for the region of interest can ensure that the adjustment of the ultrasound imaging parameters is beneficial for image quality improvement of the region of interest. In addition, some ultrasound imaging parameters capable of improving the overall ultrasound image quality may have adverse impacts such as excessive consumption of processor resources and generation of a large amount of heat, and dedicating these ultrasound imaging parameters to the region of interest can save resources as much as possible, avoid adverse impacts such as overheating, and further improve the image quality of the region of interest.

In addition, the embodiments of the present application further allow for certain deviation for a region requiring ultrasound imaging parameter optimization relative to the region of interest. For example, optimizing the ultrasound imaging parameters for at least part of the region of interest means allowing for a case in which the region of interest may have a partial region incapable of being fully optimized. This may depend on factors such as the size of the region of interest, whether adjusted parameters can satisfy the entire region, etc. Skipping optimizing the imaging parameters for at least part of the ultrasound image outside the region of interest means that a part of the ultrasound image outside the region of interest may also be optimized. This is partly because the embodiments of the present application allow for certain deviation in the optimization, and partly due to the nature of the ultrasound imaging method itself. For example, in a volumetric imaging process using a mechanical 4D probe, an ultrasound transducer performs a sector-shaped scan within a housing of the probe. When the ultrasound imaging parameters need to be optimized for the region of interest, for example, when a frame frequency needs to be increased for the region, it is inevitable that the line of sight on the ultrasound image in an entire depth direction including the region of interest will be optimized. This case should be allowed.

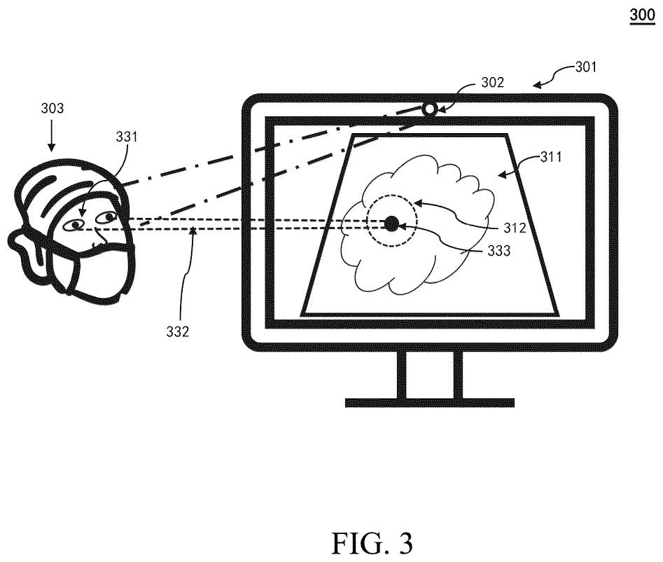

For clearer description of the technical solutions of the present application, refer to FIG. 3, which shows a schematic diagram 300 of determining a user's region of interest on an ultrasound image according to some embodiments of the present application.

As shown in FIG. 3, an image acquisition unit 302 is integrated on a display 301. In some embodiments, the image acquisition unit 302 may be fixedly arranged on the display 301. In this way, the degree of device integration is increased. Moreover, regardless of a change in the position of the display 301 relative to the user, both the position of the display 301 relative to an eye and the position of the image acquisition unit 302 relative to the eye can always maintain a good correspondence, thereby improving the accuracy of determining the user's region of interest. In addition, although FIG. 3 shows one image acquisition unit 302, in another embodiment, there may be multiple image acquisition units 302. The multiple image acquisition units simultaneously capture images of the user's eye, thereby providing richer information for determining the user's region of interest and ensuring more accurate determination.

The image acquisition unit 302 is configured to acquire image information of a surrounding environment. For example, the image acquisition unit 302 may capture an image within a certain range in front of the display 301. Usually, a user 303 needs to observe an ultrasound image 311 on the display 301 at a certain distance in front of the display 301. In this case, the image acquisition unit 302 can capture an image including the user 303 and an eye 331 of the user 303 and send the image to a processor (not shown). In some examples, the direction of the user's line of sight 332 may be determined by using a pre-stored algorithm. For example, the direction of the line of sight 332 may be determined on the basis of information in various aspects such as the orientation of the center of the eye and the position of a pupil. The direction of the line of sight 332 may be understood as the direction (or the direction and the distance) of the line of sight 332 relative to the image acquisition unit 302. Information such as the relative position and direction between the image acquisition unit 302 and the display 301 is preset. In this case, the user's point of interest 333 on the ultrasound image 311 can be determined on the basis of the line of sight direction 332.

It should be understood that the line of sight detection can be detected by a variety of means. In an embodiment, the user's line of sight being detected includes detecting a position on the ultrasound image towards which the user's line of sight is directed. That is, while the image acquisition unit 302 acquires an environment image in real time, an algorithm detects the user's position of interest on the ultrasound image in real time. Once the position of interest is determined, ultrasound image optimization is performed on the user's region of interest. In this way, the response speed of a device can be increased, ensuring that the image quality of the user's region of interest is promptly improved. In a further embodiment, the user's line of sight being detected includes detecting a position on the ultrasound image towards which the user's line of sight is directed and the duration for which the line of sight is on the position. That is, in addition to determining the user's position of interest on the ultrasound image, the duration of continuous interest in the position is also determined. For example, a time threshold may be set, and a position of a certain ultrasound image is regarded as the region of interest, and ultrasound imaging optimization is performed only when the user is continuously interested in the position for more than the time threshold (for example, three seconds). Such a configuration manner can reduce the amount of the computation of the device while avoiding mishandling of ultrasound imaging parameters. This configuration manner is particularly suitable for a use scenario in which the user quickly browses ultrasound images. It should be understood that the foregoing different manners of detecting the user's line of sight may be automatically set by a machine by default, or may be selected by the user. For example, the user may choose to switch between different modes or adjust the time threshold.

With continued reference to FIG. 3, the user's region of interest 312 on the ultrasound image 311 can be automatically determined by detecting the user's line of sight 332. In an example, the automatic determination of the region of interest 312 may be as follows: determining the user's point of interest 333 on the ultrasound image 311 on the basis of the detection of the user's line of sight 332; and automatically determining a certain range of ultrasound image around the point of interest 333 as the region of interest 312.

In this way, it is possible to quickly determine a position that needs optimization and adjustment for ultrasound imaging parameters, with no need for an additional complex data processing process. The shape and the size of the region of interest 312 may be adjusted according to multiple circumstances. For example, the shape and the size may be selected according to various factors such as the size of the display 301 or the size of the ultrasound image 311. In addition, in some embodiments, the shape and the size described above may also be adjusted by the user.

In a further example, the automatic determination of the region of interest may be as follows: determining the user's point of interest on the ultrasound image on the basis of the detection of the user's line of sight; performing image recognition on an ultrasound image of the point of interest, and determining a corresponding anatomical feature of the point of interest; and automatically determining an entire ultrasound image of the corresponding anatomical feature as the region of interest. The foregoing image recognition manner may be any image recognition manner in existing technologies, or may be implemented by the embodiments described above of the present application, and details will not be described herein again.

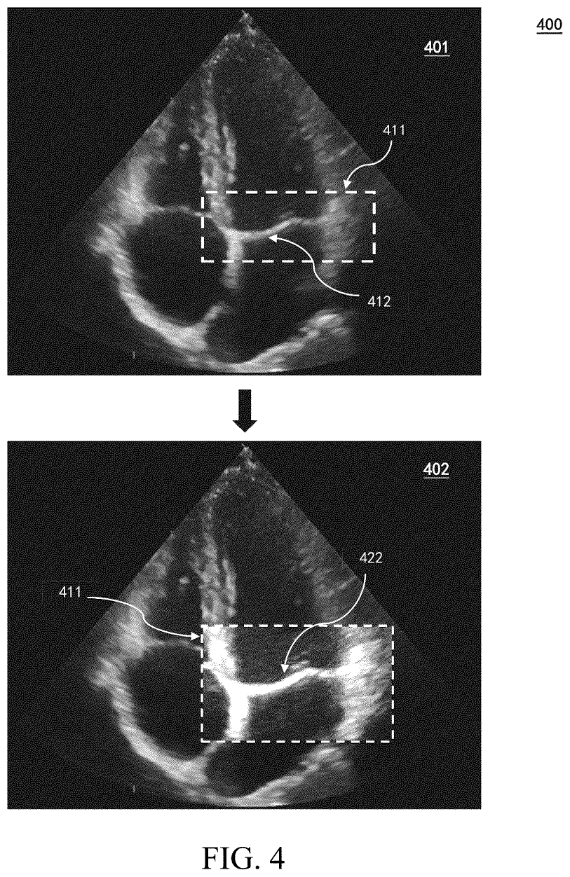

Such a manner can accurately determine an anatomical feature that the user is currently interested in, and is more suitable for clinical use. An exemplary description is provided with reference to FIG. 4. FIG. 4 shows a schematic diagram 400 of optimizing an ultrasound image according to some embodiments of the present application. In an image 401, a user's point of interest (not shown) lies in a local portion of a mitral valve 412 (an anatomical feature). At this time, it can be determined by using image recognition technology that an anatomical feature corresponding to the point of interest is the mitral valve 412. Further, an entire ultrasound image of the mitral valve may be automatically set as a region of interest 411. In this case, the user can obtain a high-quality image 402 of the entire mitral valve 412 during imaging and observation of the mitral valve 422. For example, a mitral valve 422 in an image 402 has higher brightness, higher resolution, etc., than the mitral valve 412 in the non-optimized image 401, and thus is more convenient for the user to observe.

In the foregoing description, the mitral valve is used as an example of the anatomical feature. In practice, the anatomical feature may alternatively be any other type. An anatomical feature may be understood as a local organ, tissue, or lesion having clinical significance. In addition, it should be noted that FIG. 3 does not show other components of a specific ultrasound imaging system, such as a probe, a host computer, a processor, etc., and the ultrasound imaging system and the method thereof according to any embodiment of the present application are applicable to each other and may be combined unless otherwise specified.

Identifying the anatomical feature corresponding to the point of interest not only can help determine the region of interest, but also can further help optimize the ultrasound imaging parameters in some embodiments. In particular, on the basis of identifying the corresponding anatomical feature, ultrasound imaging parameters being optimized for the region of interest includes configuring the ultrasound imaging parameters for the corresponding anatomical feature. A detailed description is provided below by using some specific embodiments.

In some embodiments, the ultrasound image of the region of interest may be automatically enlarged and displayed in response to the corresponding anatomical feature being small in size in a current ultrasound image. A detailed description is provided with continued reference to FIG. 4. Firstly, in the image 401, it is determined by means of line-of-sight detection and image recognition that the region of interest 411 includes the mitral valve 412. Further, specific optimization may be performed for the anatomical feature, namely, the mitral valve 412, and the optimized image 402 is obtained. Considering that the mitral valve 412 is small in size in the current ultrasound image, the mitral valve 422 is enlarged and displayed in the image 402. In this case, a doctor can observe the mitral valve 422 more clearly. The embodiments are also applicable to other anatomical features having very small sizes, e.g., capillaries and the like. In addition, the image 402 shows a case in which the enlarged and displayed image of the mitral valve 422 is overlaid on the image 402. In a further embodiment, instead of overlaying, distortion processing can be applied to the image 402, allowing the entire ultrasound image to be displayed while the region of interest is enlarged.

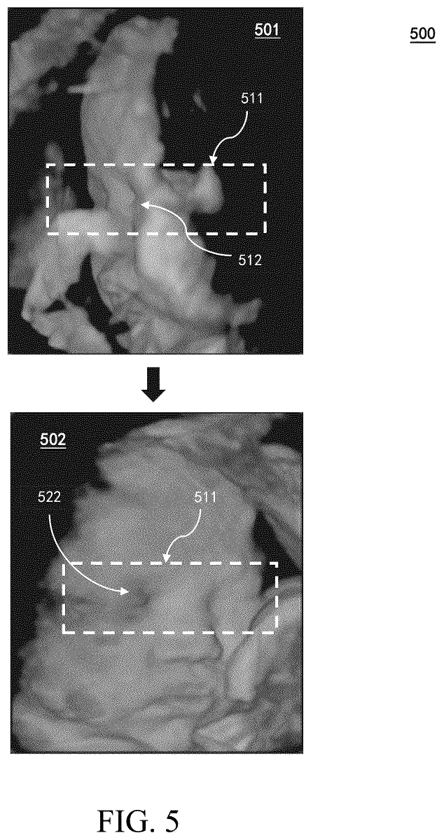

In a further example, an optimized angle of view may be further automatically displayed for the corresponding anatomical feature in response to the ultrasound image being a volumetric image. A detailed description is provided with reference to FIG. 5. FIG. 5 shows a schematic diagram 500 of optimizing a volumetric ultrasound image according to some other embodiments of the present application. Firstly, in an image 501, it is determined by means of line-of-sight detection and image recognition that a region of interest 511 includes an anatomical feature, namely, a fetal eye 512. However, as shown in the image 501, an image angle of view of the fetal eye 512 is occluded in the image 501 and cannot be fully observed. Conventionally, a user needs to move the position of the probe or perform adjustment by operating a user interface to obtain a satisfactory angle of view. In the embodiments, after identifying that the region of interest 511 includes the anatomical feature (the fetal eye 512), an optimized angle of view can be automatically displayed for the anatomical feature, thereby facilitating observation. For example, as shown in an image 502, a fetal eye 522 has been adjusted to obtain an angle of view more favorable for observation by the user.

The two examples above are exemplary descriptions of targeted ultrasound imaging parameter optimization performed for an identified anatomical feature. Automatic optimization may alternatively be performed in another manner on the basis of determining a type of anatomical feature. In addition, it should be noted that the optimization manner described in the foregoing embodiments and the optimization for any other ultrasound imaging parameters described above in the present application are not contradictory. In some embodiments, multiple optimization manners for the ultrasound imaging parameters may be selected. For example, one may choose to perform optimization for general ultrasound imaging parameters, such as enlarging and displaying an anatomical feature, increasing resolution, and increasing probe power (for example, in the example in FIG. 4, the mitral valve is enlarged and other imaging parameters are also optimized).

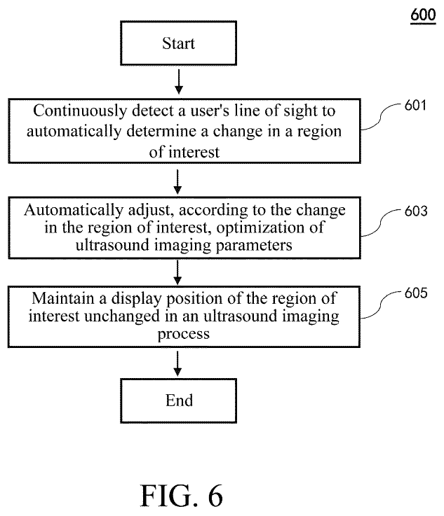

The ultrasound imaging process is a real-time, dynamic process. To further improve work efficiency and user experience in the real-time imaging process, some embodiments of the present application further propose improvements. Referring to FIG. 6, this figure shows a flowchart of an ultrasound imaging method 600 according to some embodiments of the present application.

In step 601, a user's line of sight is continuously detected to automatically determine a change in a region of interest. A manner of detecting the user's line of sight and a method for automatically determining the region of interest may be those described in any embodiment above of the present application, and details will not be described herein again. The user needs to observe different ultrasound section images in an ultrasound scanning process, and may observe different positions even for the same image. Therefore, the region of interest may be constantly changing. In the step of the present application, a processor continuously detects and analyzes continuity of the line of sight, to determine whether the region of interest has changed, and/or determine a specific position to which the region of interest has changed.

In step 603, optimization of ultrasound imaging parameters is automatically adjusted according to the change in the region of interest. According to the change in the region of interest, the processor may determine whether there is a new change region. If so, the ultrasound imaging parameters are adjusted to optimize the new region of interest. An optimization manner may be that described in any embodiment above of the present application. For example, general parameters (e.g., a frame frequency, resolution, a probe power, etc.) capable of improving ultrasound image quality may be configured for the region of interest. Ultrasound imaging parameters (e.g., parameters such as a focal point, a depth, time gain compensation, etc.) may also be optimized for the region of interest. Alternatively, image recognition may also be performed on the region of interest, to determine an anatomical feature of the new region of interest according to an image recognition result, and provide special ultrasound imaging parameter optimization for the anatomical feature.

Such a configuration manner enables an ultrasound imaging system to optimize ultrasound image quality in a way that is more user-friendly and better meets the user's preferences. As the user's region of interest changes constantly, the ultrasound imaging parameter optimization changes accordingly. On the one hand, it is ensured that the user can observe a high-quality image at any position at any time. On the other hand, since the ultrasound imaging parameter optimization is mainly directed towards the region of interest rather than the entire ultrasound image, resources of the ultrasound imaging system are not excessively wasted and occupied, thereby ensuring that high-performance stable operation of the system can be sustainably maintained.

In addition, it is further recognized by the inventor that, in an actual ultrasound imaging process, a user may need to move a probe or perform other operations to ensure that optimal ultrasound data is acquired for images of the same anatomical position. Moving the ultrasound probe inevitably leads to a change in a field of view. Accordingly, a position in an ultrasound image displayed on a display is also changed. To further improve the user's convenience in ultrasound imaging, some embodiments of the present application further propose improvements.

Optionally, in some other embodiments of the present application, the method may further include step 605: maintaining a display position of the region of interest unchanged in the ultrasound imaging process. That is, once the region of interest is determined, the processor will maintain the position of the region of interest on the display unchanged. In this way, when the user needs to observe a certain region of interest but also needs to perform another operation, for example, moving the ultrasound probe, the region of interest is fixed at a specific position on the display. This allows the user to observe without having to move the line of sight thereof.

In some embodiments, step 605 may be used together with steps 601 and 603 as one complete implementation. For example, in a complete workflow, when the user's region of interest is at a specific position, an ultrasound image of the region of interest is fixed at the specific position on the display. Even if the user moves the probe, the ultrasound image of the region of interest does not move. This can ensure stability of the ultrasound image observed by the user. From the perspective of the user's intuitive experience, the user's intuitive experience will not be affected by a change in the field of view of the ultrasound image because the ultrasound image is fixed. The user can perceive a change in quality of the ultrasound image caused by moving the probe, thereby making it easier to obtain a satisfactory image. When the user's observation on a previous region of interest ends and the position of the region of interest is changed, ultrasound imaging parameters for a new region of interest are first optimized for improvement. Subsequently, the position of the new region of interest will also be fixed at a specific position on the display, and the user can perform an operation such as moving the probe to intuitively experience further changes in the image quality. In a further embodiment, step 605 may be implemented independently of steps 601 and 603, for example, it may be combined with any embodiment described above of the present application.

Further, some embodiments of the present application further provide an ultrasound imaging system. The ultrasound imaging system includes:
a probe, configured to acquire ultrasound data about tissue to be imaged;
a processor, configured to perform the method according to any embodiment of the present application;
a display, the display receiving a signal from the processor and performing display; and
an image acquisition unit, configured to acquire a user's image and send the image to the processor.

The probe, the display, and the image acquisition unit in the ultrasound imaging system may be those described in any embodiment of this application. For example, references can be made to the description of the ultrasound imaging system shown in FIG. 1. Alternatively, the probe, the display, and the image acquisition unit may also be any other types in the art. The processor may also be the processor in the controller circuit in FIG. 1. The method performed by the processor may be that described in any embodiment above of the present application, and the method steps in any embodiment above of the present application may be combined in any manner unless specifically excluded.

Some embodiments of the present application further provide a non-transitory computer-readable medium, wherein the non-transitory computer-readable medium has a computer program stored therein, the computer program has at least one code segment, and the at least one code segment is executable by a machine so as to enable the machine to execute the steps of the method in any of the above embodiments.

Correspondingly, the present disclosure may be implemented by means of hardware, software, or a combination of hardware and software. The present disclosure may be implemented in at least one computer system in a centralized manner, or implemented in a distributed manner; and in the distributed manner, different elements are distributed on a plurality of interconnected computer systems. Any type of computer system or other apparatus suitable for implementing the methods described herein is considered to be appropriate.

Various embodiments may also be embedded in a computer program product, which includes all features capable of implementing the methods described herein, and the computer program product is capable of executing these methods when loaded into a computer system. The computer program in this context means any expression in any language, code, or symbol of an instruction set intended to enable a system having information processing capabilities to execute a specific function directly or after any or both of the following: a) conversion to another language, code, or symbol; and b) replication in different material forms.

The purpose of providing the above specific embodiments is to facilitate understanding of the content disclosed in the present invention more thoroughly and comprehensively, but the present invention is not limited to these specific embodiments. Those skilled in the art should understand that various modifications, equivalent replacements, and changes can also be made to the present invention and should be included in the scope of protection of the present invention as long as these changes do not depart from the spirit of the present invention.

1. An ultrasound imaging system comprising:
an ultrasound probe comprising:
a transducer configured to transmit and receive an ultrasound signal;
a matching layer configured to have an acoustic impedance between a tissue to be imaged and a material of the transducer; and
a damping block configured to absorb ultrasound energy;
a processing circuit having a processor coupled to a memory device storing instructions thereon that, when executed, cause the processing circuit to perform operations comprising:
generating and displaying an ultrasound image of tissue to be imaged;
detecting a user's line of sight, and automatically determining the user's region of interest on the ultrasound image; and
optimizing ultrasound imaging parameters for the region of interest, and generating and displaying an optimized ultrasound image for the region of interest.

2. The system according to claim 1, wherein the detecting a user's line of sight comprises:
detecting a position on the ultrasound image towards which the user's line of sight is directed, or detecting a position on the ultrasound image towards which the user's line of sight is directed and the duration for which the line of sight is on the position.

3. The system according to claim 1, wherein the optimizing ultrasound imaging parameters for the region of interest comprises:
optimizing ultrasound imaging parameters for at least part of the region of interest, and skipping optimizing imaging parameters for at least part of the ultrasound image outside the region of interest.

4. The system according to claim 1, wherein the ultrasound imaging parameters comprise at least one of ultrasound data acquisition-related parameters and ultrasound image processing-related parameters.

5. The system according to claim 1, wherein the automatic determination of the region of interest comprises:
determining the user's point of interest on the ultrasound image based on the detection of the user's line of sight; and
automatically determining a certain range of ultrasound image around the point of interest as the region of interest.

6. The system according to claim 1, wherein the automatic determination of the region of interest comprises:
determining the user's point of interest on the ultrasound image on the basis of the detection of the user's line of sight;
performing image recognition on an ultrasound image of the point of interest, and determining a corresponding anatomical feature of the point of interest; and
automatically determining an entire ultrasound image of the corresponding anatomical feature as the region of interest.

7. The system according to claim 6, wherein the optimizing ultrasound imaging parameters for the region of interest comprises configuring the ultrasound imaging parameters for the corresponding anatomical feature.

8. The system according to claim 7, wherein the configuring the ultrasound imaging parameters for the corresponding anatomical feature comprises at least one of the following manners:
automatically enlarging and displaying the ultrasound image of the region of interest in response to the corresponding anatomical feature being smaller than a predefined size; and
automatically displaying an optimized angle of view for the corresponding anatomical feature in response to the ultrasound image being a volumetric image.

9. The system according to claim 1, wherein the processing circuit is further configured to perform operations comprising:
continuously detecting the user's line of sight to automatically determine a change in the region of interest; and
automatically adjusting, according to the change in the region of interest, optimization of the ultrasound imaging parameters.

10. The system according to claim 1, wherein the processing circuit is further configured to perform operations comprising:
maintaining a display position of the region of interest unchanged in the ultrasound imaging process.

11. An ultrasound imaging method, comprising:
generating and displaying an ultrasound image of tissue to be imaged;
detecting a user's line of sight, and automatically determining the user's region of interest on the ultrasound image; and
optimizing ultrasound imaging parameters for the region of interest, and generating and displaying an optimized ultrasound image for the region of interest.

12. The method according to claim 11, wherein the detecting a user's line of sight comprises:
detecting a position on the ultrasound image towards which the user's line of sight is directed, or detecting a position on the ultrasound image towards which the user's line of sight is directed and the duration for which the line of sight is on the position.

13. The method according to claim 11, wherein the optimizing ultrasound imaging parameters for the region of interest comprises:
optimizing ultrasound imaging parameters for at least part of the region of interest, and skipping optimizing imaging parameters for at least part of the ultrasound image outside the region of interest.

14. The method according to claim 11, wherein the ultrasound imaging parameters comprise at least one of ultrasound data acquisition-related parameters and ultrasound image processing-related parameters.

15. The method according to claim 11, wherein the automatic determination of the region of interest comprises:
determining the user's point of interest on the ultrasound image based on the detection of the user's line of sight; and
automatically determining a certain range of ultrasound image around the point of interest as the region of interest.

16. The method according to claim 11, wherein the automatic determination of the region of interest comprises:
determining the user's point of interest on the ultrasound image on the basis of the detection of the user's line of sight;
performing image recognition on an ultrasound image of the point of interest, and determining a corresponding anatomical feature of the point of interest; and
automatically determining an entire ultrasound image of the corresponding anatomical feature as the region of interest.

17. The method according to claim 16, wherein the optimizing ultrasound imaging parameters for the region of interest comprises configuring the ultrasound imaging parameters for the corresponding anatomical feature, and
wherein the configuring the ultrasound imaging parameters for the corresponding anatomical feature comprises at least one of the following manners:
automatically enlarging and displaying the ultrasound image of the region of interest in response to the corresponding anatomical feature being smaller than a predefined size in a current ultrasound image; and
automatically displaying an optimized angle of view for the corresponding anatomical feature in response to the ultrasound image being a volumetric image.

18. The method according to claim 11, further comprising:
continuously detecting the user's line of sight to automatically determine a change in the region of interest; and
automatically adjusting, according to the change in the region of interest, optimization of the ultrasound imaging parameters.

19. The method according to claim 11, further comprising:
maintaining a display position of the region of interest unchanged in the ultrasound imaging process.

20. A non-transitory computer-readable medium, wherein the non-transitory computer-readable medium has a computer program stored therein, the computer program has at least one code segment, and the at least one code segment is executable by a machine to enable the machine to perform the operations of:
generating and displaying an ultrasound image of tissue to be imaged;
detecting a user's line of sight, and automatically determining the user's region of interest on the ultrasound image; and
optimizing ultrasound imaging parameters for the region of interest, and generating and displaying an optimized ultrasound image for the region of interest.
